(19) United States
(12) Patent Application Publication
(10) Pub. No.: US 2023/0225596 A1
(43) Pub. Date: Jul. 20, 2023

(54) SHAPE MEMORY ENDOSCOPE INSERTION TUBE SHEATH

(71) Applicant: Nitesh Ratnakar, Wheeling, WV (US)

(72) Inventor: Nitesh Ratnakar, Wheeling, WV (US)

(21) Appl. No.: 17/117,102

(22) Filed: Dec. 9, 2020

Related U.S. Application Data
(60) Provisional application No. 62/945,863, filed on Dec. 9, 2019.

(51) Int. Cl. A61B 1/005; A61B 1/00

(52) U.S. Cl. CPC A61B 1/0058; A61B 1/00078

(57) ABSTRACT

Presently disclosed is an endoscope comprising of an insertion tube sheath, which comprises one or more mechanical layers having a mechanical contraption, wherein one or more of the mechanical contraptions is made of shape memory material.

### CROSS-REFERENCE TO RELATED APPLICATION

This application claims the benefit under 35 U.S.C. § 119(e) of Provisional Patent Application No. 62/945,863, filed Dec. 9, 2019, the entire disclosure of which is hereby incorporated by reference.

### TECHNICAL FIELD

The present disclosure relates to endoscopes, more specifically to endoscopes with inner construction that minimizes looping during examination of a hollow body organ.

### RELATED ART

Endoscopes are used to perform a variety of surgical procedures. FIGS. 1 and 2 illustrate an implementation of a conventional endoscope. It has a handle from which extends a flexible shaft, which is inserted into a hollow organ to be inspected. The shaft consists of a proximal section, insertion tube, bending section and a stiff section. The shaft terminates in the distal end, which typically houses image lens, illumination bulb, air/water nozzle and an instrument channel outlet. Light is transmitted from a light source through the shaft via an electric cable to the illumination bulb. The illumination bulb illuminates the area to be examined. The image lens captures images of the illuminated area. The image is then transmitted through a fiber optic cable and viewed through an eyepiece on the handle of the endoscope. Alternatively, the image is converted to a video signal and transmitted to an image processor by an electrical cable. The image is then processed and displayed on a display unit like a computer monitor. The handle of the endoscope has an extension arm that attaches the endoscope to a light source and an image processor.

To enable the endoscope to maneuver through the turns of a hollow organ the shaft is flexible and incorporates a multitude of cables that attach the bending portion with actuators. Tension is applied to these cables to move the bending portion in various directions. This is done by manual adjustment of actuators on the handle of the endoscope. Typically, there are two pairs of such cables passing within the shaft, one pair for flexing the bending portion in one plane and the other pair for flexing it in an orthogonal plane.

It is also usual to provide two channels extending between the handle and the distal end of the shaft, an air/water channel and an instrument channel. The air/water channel is used to insufflate air in a hollow organ to expand it for proper visualization. The air/water channel is connected proximally to an air/water pump (not shown) and distally to the air/water channel outlet. The image lens and the illumination bulb are frequently smeared with blood, stool or other body fluids while in a hollow organ which obstructs a clear view. In such a situation, the air/water channel is used to eject water or blow air at the image lens and/or illumination bulb in order to clean them while still inside a hollow organ. The instrument channel has an inlet proximally and an outlet distally. It is used to pass various surgical instruments to do various surgical procedures. It is also used to apply suction to remove fluids, air and other materials from within a hollow organ during examination.

Endoscope is typically inserted into the patient either through a natural body orifice like anus or mouth or it is inserted through a surgical incision. It is then steered to a desired location by adjusting the bending portion and manually pushing the endoscope. After reaching the desired location, the endoscope is withdrawn. Typically, it is during pull out when the inside of a hollow organ like colon is thoroughly examined. Insertion of the endoscope into a hollow organ is a risky maneuver and is associated with significant complications like trauma, bleeding and perforation. It is generally desirable to complete the examination with a single insertion to minimize complications.

The conventional endoscopes have significant limitations. One such limitation is that the endoscope insertion tube often forms a loop when advanced through a tortuous hollow body organs such as colon. Loop formation not only causes patient discomfort but also causes procedure complications such as perforation and tissue trauma. It also results in physicians not being able to complete the procedure and sometimes significantly prolongs the procedure and anesthesia time.

### SUMMARY OF THE PRESENT DISCLOSURE

In light of the significant limitation of looping discussed above, there is a need for an endoscopic system that prevents and minimizes loop formation during passage through a hollow organ. The present disclosed systems and methods address these unmet needs.

The present disclosed systems and methods prevent and minimize loop formation by an endoscope. This is achieved by coating the inner lining of the insertion tube with one or more ‘shape memory’ material such as Nitinol. The shape memory inner lining is programmed to keep the endoscope in baseline straight position (austenite straight position) such that when the endoscope attempts to form a loop, the insertion tube shape memory inner lining counters such looping tendency by attempting to revert to its austenite straight position. In one embodiment of the present disclosure, the shape memory material and a variation of the Nitinol alloy with austenite transformation temperature that is around 72° F. (room temperature).

Additional features and advantages of the present disclosure will be set forth in the description and drawings which follow or may be learned by practice of the presently disclosed systems and methods.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

FIG. 1 shows a view of a conventional endoscope.

FIG. 2 shows a side view of the distal end, bending section and insertion tube of a conventional endoscope.

FIG. 3A shows a schematic side view of an insertion tube of a conventional endoscope.

FIG. 3B shows a schematic side view of an insertion tube sheath of a conventional endoscope.

A conventional endoscope may be inside a colon in a straight position.

A conventional endoscope may be inside a colon in a looped position.

A conventional endoscope may be inside a colon in a looped position causing patient discomfort.

A conventional endoscope may be inside a colon in a looped position causing colon perforation.

FIGS. 4A-F show exemplary embodiments of the present disclosure.

FIG. 5 shows a few possible mechanical configurations of the ‘exemplary Nitinol” mesh.

The endoscope of the present disclosure may be operated in conventional settings inside a colon, including: (a) in a straight position; (b) attempting to form a loop and its opposition to the looping tendency due to shape memory inner lining; and c) straightening due to the workings of the shape memory inner lining.

FIGS. 6A-C illustrates some unique properties of Nitinol.

### DETAIL DESCRIPTION

In the following detailed description of exemplary embodiments of the disclosure in this section, specific exemplary embodiments in which the disclosure may be practiced are described in sufficient detail to enable those skilled in the art to practice the disclosed embodiments. However, it is to be understood that the specific details presented need not be utilized to practice embodiments of the present disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one embodiment of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various requirements are described which may be requirements for some embodiments but not other embodiments.

Those of ordinary skill in the art will appreciate that the components and basic configuration depicted in the following figures may vary. Other similar or equivalent components may be used in addition to or in place of the components depicted. A depicted example is not meant to imply limitations with respect to the presently described one or more embodiments and/or the general disclosure.

In an exemplary embodiment of the present disclosure, Nitinol is used as the shape memory material. More specifically, a Nitinol alloy with austenite finish temperature of about 72° F. is used. Nitinol's unusual properties are derived from a reversible solid-state phase transformation known as a martensitic transformation, between two different martensite crystal phases, requiring 10.000-20,000 psi (69-138 MPa) of mechanical stress.

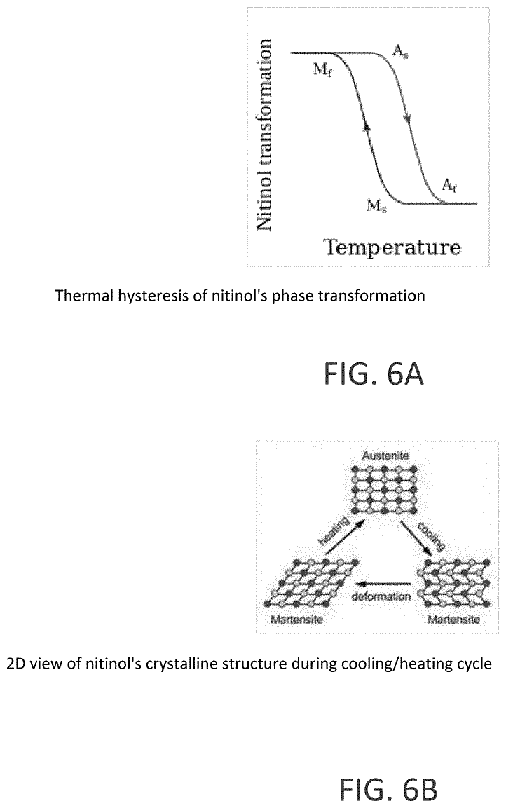

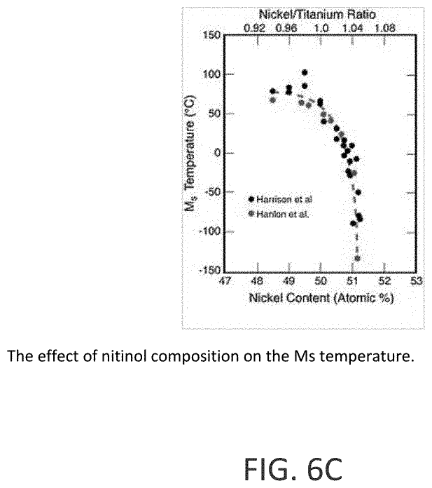

FIGS. 6A-6C demonstrate some unique properties of Nitinol. At high temperatures, nitinol assumes an interpenetrating simple cubic structure referred to as austenite (also known as the parent phase). At low temperatures, nitinol spontaneously transforms to a more complicated monoclinic crystal structure known as martensite (daughter phase)¹. There are four transition temperatures associated to the austenite-to-martensite and martensite-to-austenite transformations. Starting from full austenite, martensite begins to form as the alloy is cooled to the so-called martensite start temperature, or Mₛ, and the temperature at which the transformation is complete is called the martensite finish temperature, or M_{f}. When the alloy is fully martensite and is subjected to heating, austenite starts to form at the austenite start temperature. A_{f}, and finishes at the austenite finish temperature. A_{f}.² ¹ See Otsuka, K.; Ren, X. (2005). “Physical Metallurgy of Ti—Ni-based Shape Memory Alloys”. Progress in Materials Science. 50 (5): 511-678. CiteSeerX 10.1.1.455.1300. doi:10.1016/j.pmatsci.2004.10.001. This reference is hereby incorporated by reference in its entirety.² See “Nitinol facts”. Nitinol.com. 2013. This reference is hereby incorporated by reference in its entirety.

The cooling/heating cycle shows thermal hysteresis. The hysteresis width depends on the precise nitinol composition and processing. Its typical value is a temperature range spanning about 20-50 K (20-50° C.; 36-90° F.) but it can be reduced or amplified by alloying³ and processing⁴. ³ See Chluba, Christoph; Ge, Wenwei; Miranda, Rodrigo Lima de; Strobel, Julian; Kienle, Lorenz; Quandt, Eckhard; Wuttig, Manfred (2015-05-29). “Ultralow-fatigue shape memory alloy films”. Science. 348 (6238): 1004-1007. Bibcode:2015Sci . . . 348.1004C. doi:10.1 126/science.1261164. ISSN 0036-8075. PMID 26023135. S2CID 2563331. This reference is hereby incorporated by reference in its entirety.⁴ See Spini, Tatiana Sobottka; Valarelli, Fabrfcio Pinelli; Caneado, Rodrigo Hermont; Freitas, Karina Maria Salvatore de; Villarinho, Denis Jardim; Spini, Tatiana Sobottka; Valarelli, Fabrfcio Pinelli; Caneado, Rodrigo Hermont; Freitas, Karina Maria Salvatore de (2014-04-01). “Transition temperature range of thermally activated nickel-titanium archwires”. Journal of Applied Oral Science. 22 (2): 109-117. doi:10.1590/1678-775720130133. ISSN 1678-7757. PMC 3956402. PMID 24676581. This reference is hereby incorporated by reference in its entirety.

Crucial to nitinol properties are two key aspects of this phase transformation. First is that the transformation is “reversible”, meaning that heating above the transformation temperature will revert the crystal structure to the simpler austenite phase. The second key point is that the transformation in both directions is instantaneous.

Martensite's crystal structure (known as a monoclinic, or B19′ structure) has the unique ability to undergo limited deformation in some ways without breaking atomic bonds. This type of deformation is known as twinning, which consists of the rearrangement of atomic planes without causing slip, or permanent deformation. It is able to undergo about 6-8% strain in this manner. When martensite is reverted to austenite by heating, the original austenitic structure is restored, regardless of whether the martensite phase was deformed. Thus the name “shape memory” refers to the fact that the shape of the high temperature austenite phase is “remembered,” even though the alloy is severely deformed at a lower temperature.⁵ ³ See Funakubo. Hiroyasu (1984). Shape memory alloys. University of Tokyo, pp. 7. 176. This reference is hereby incorporated by reference in its entirety.

A great deal of pressure can be produced by preventing the reversion of deformed martensite to austenite—from 35.000 psi to, in many cases, more than 100.000 psi (689 MPa). One of the reasons that nitinol works so hard to return to its original shape is that it is not just an ordinary metal alloy, but what is known as an intermetallic compound. In an ordinary alloy, the constituents are randomly positioned in the crystal lattice; in an ordered intermetallic compound, the atoms (in this case, nickel and titanium) have very specific locations in the lattice.⁶ The fact that nitinol is an intermetallic is largely responsible for the complexity in fabricating devices made from the alloy. ⁶ See “Nitinol SM495 Wire” (PDF). 2013. Archived from the original (properties, PDF) on 2011 Jul. 14. This reference is hereby incorporated by reference in its entirety.

The scenario described above (cooling austenite to form martensite, deforming the martensite, then heating to revert to austenite, thus returning the original, undeformed shape) is known as the thermal shape memory effect. To fix the original “parent shape.” the alloy must be held in position and heated to about 500° C. (932° F.). This process is usually called shape setting. A second effect, called superelasticity or pseudoelasticity, is also observed in nitinol. This effect is the direct result of the fact that martensite can be formed by applying a stress as well as by cooling. Thus, in a certain temperature range, one can apply a stress to austenite, causing martensite to form while at the same time changing shape. In this case, as soon as the stress is removed, the nitinol will spontaneously return to its original shape. In this mode of use, nitinol behaves like a super spring, possessing an elastic range 10-30 times greater than that of a normal spring material. There are, however, constraints: the effect is only observed about 273-313 K (0-40° C.; 0-72° F.) above the A_{f} temperature. This upper limit is referred to as M_{d}, which corresponds to the highest temperature in which it is still possible to stress-induce the formation of martensite. Below M_{d}, martensite formation under load allows superelasticity due to twinning. Above M_{d}, since martensite is no longer formed, the only response to stress is slip of the austenitic microstructure, and thus permanent deformation.

Nitinol is typically composed of approximately 50 to 51% nickel by atomic percent (55 to 56% weight percent).⁷ Making small changes in composition can change the transition temperature of the alloy significantly. Transformation temperatures in nitinol can be controlled to some extent, where A_{f} temperature ranges from about −20° C. to +110° C. Thus, it is common practice to refer to a nitinol formulation as “superelastic” or “austenitic” if A_{f} is lower than a reference temperature, while as “shape memory” or “martensitic” if higher. The reference temperature is usually defined as the room temperature or human body temperature (37° C.; 98° F.). See “Nitinol SE508 Wire” (PDF). 2013. Archived from the original (properties, PDF) on 2011 Jul. 14. This reference is hereby incorporated by reference in its entirety. Also see footnote 6.

One often-encountered effect regarding nitinol is the so-called R-phase. The R-phase is another martensitic phase that competes with the martensite phase mentioned above. Because it does not offer the large memory effects of the martensite phase, it is usually of non-practical use.

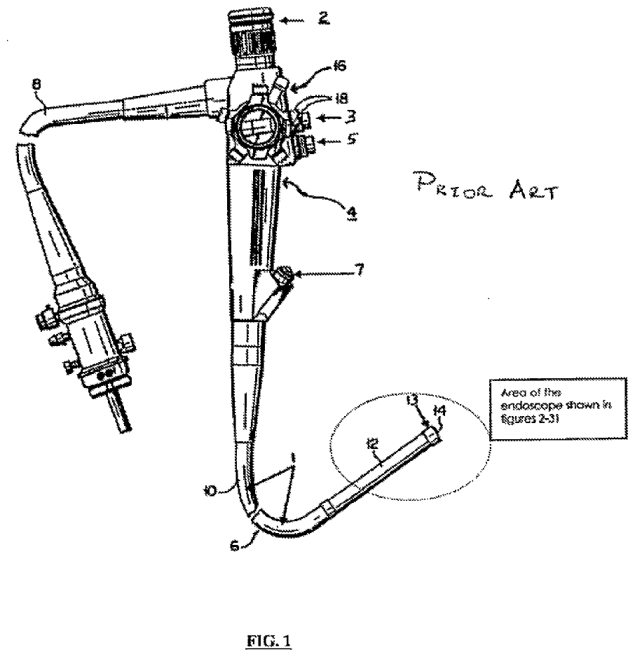

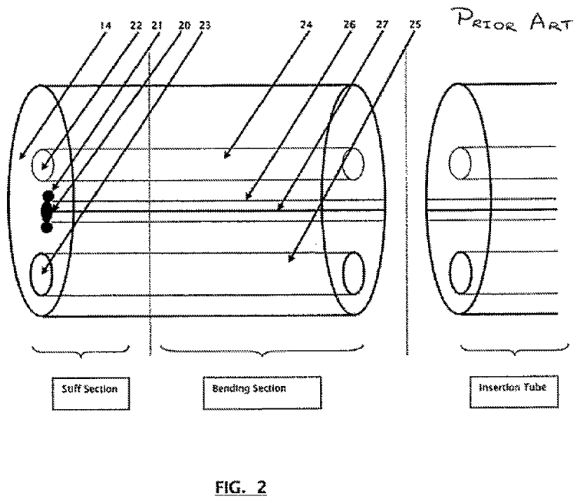

FIGS. 1 and 2 illustrate an example of a conventional endoscope. It has a handle (4) from which extends a flexible shaft (1), which is inserted into a hollow organ to be inspected. The shaft consists of a proximal section (10), insertion tube (6), bending section (12) and a stiff section (13). The insertion tube is covered with an insertion tube sheath. The shaft terminates in the distal end (14), which typically houses one image lens (20), one to two illumination bulbs (21), air/water nozzle (22) and an instrument channel outlet (23). Light is transmitted from a light source through the shaft via an electric cable (26) to the illumination bulb (21). The illumination bulb illuminates the area to be examined. The image lens (20) captures images of the illuminated area. The image is then transmitted through a fiber optic cable (27) and viewed through an eyepiece (2) attached to the handle of the endoscope. Alternatively, the image is converted to a video signal and is then transmitted to an image processor by an electrical cable. The image is processed and displayed on a display unit like a computer monitor (not shown). The handle (4) of the endoscope has a grip (16) and an extension arm (8) that attaches the endoscope to a light source and an image processor.

To enable the endoscope to maneuver through the turns of a hollow organ the shaft is flexible and incorporates a multitude of wires that attach the bending portion (12) with actuators (18). Typically, there are two pairs of such wires passing within the shaft, one pair for flexing the bending portion in one plane and the other pair for flexing it in an orthogonal plane. Tension is applied to these wires using the actuators (18) to move the bending portion (12) in various directions.

It is also usual to provide two channels extending between the handle and the distal end of the shaft, an air/water channel (24) and an instrument channel (25). The air/water channel (24) is used to insufflate air in a hollow organ to expand it for proper visualization. The air/water channel is connected proximally to an air/water pump (not shown) and to distally to the air/water nozzle (22). It is controlled by an air/water control valve (5) located on the handle (4). The image lens (20) and the illumination bulb (21) are frequently smeared with blood, stool or other body fluids while in a hollow organ. In such a situation, the air/water channel (24) is used to squirt water or blow air at the image lens (20) and/or illumination bulb (21) in order to clean them while still inside a hollow organ. The instrument channel (25) has an instrument channel inlet (7) proximally and an instrument channel outlet (23) distally. It is used to pass surgical instruments to do various surgical procedures. It is also used to apply suction using the suction control valve (3) located on the handle (4). This suction is useful in removing fluids, air and other materials from within a hollow organ during examination.

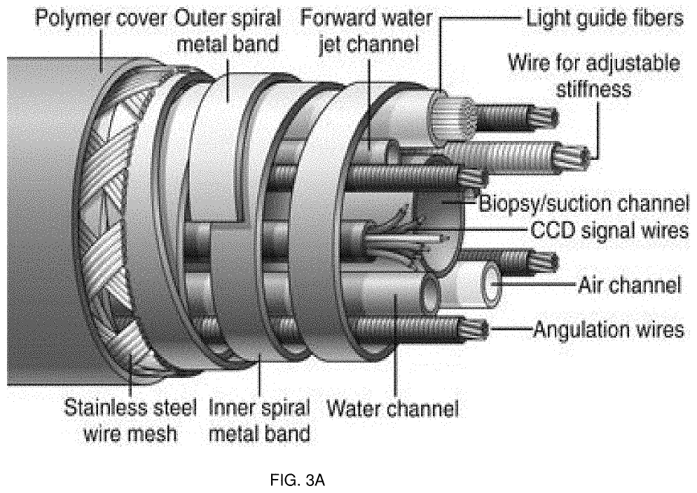

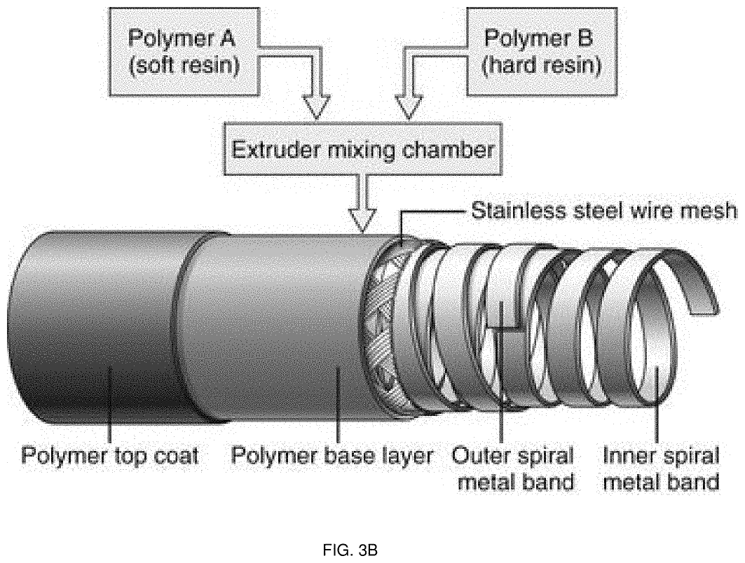

FIG. 3A and FIG. 3B show the details of the insertion tube sheath. It comprises a flexible straight hollow tube comprising of several layers of flexible materials a) inner spiral band b) outer spiral band c) stainless steel wire mesh d) polymer base layer e) polymer top coat. All these layers are mechanically designed to be flexible to aid in insertion of the endoscope through hollow body organs.

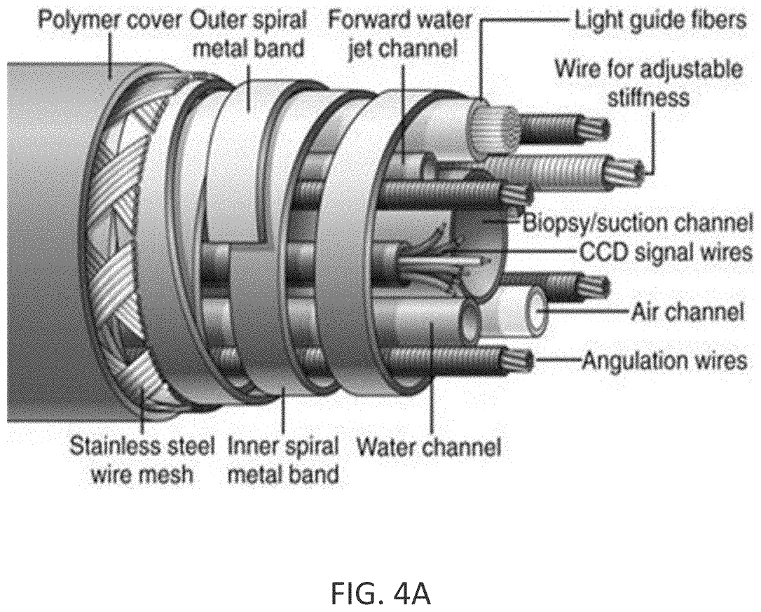

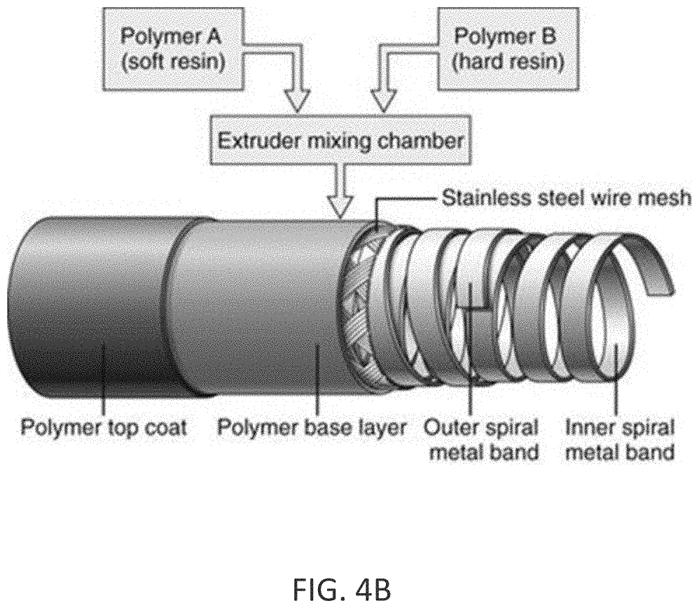

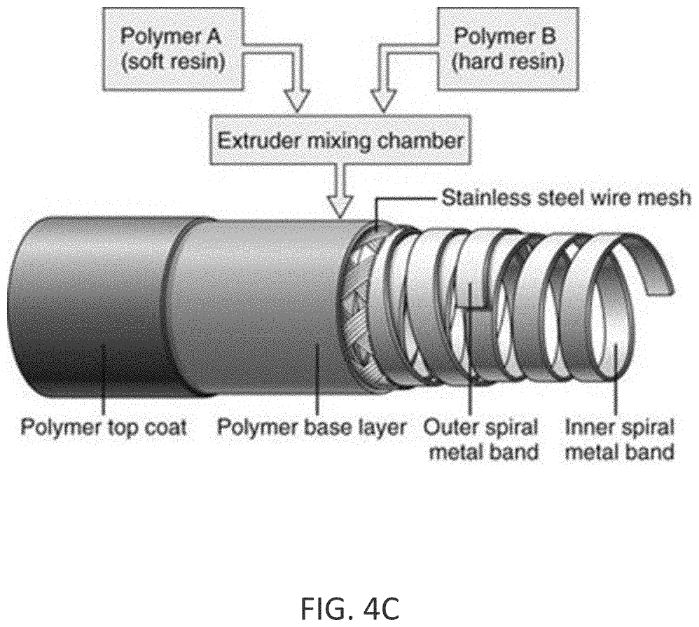

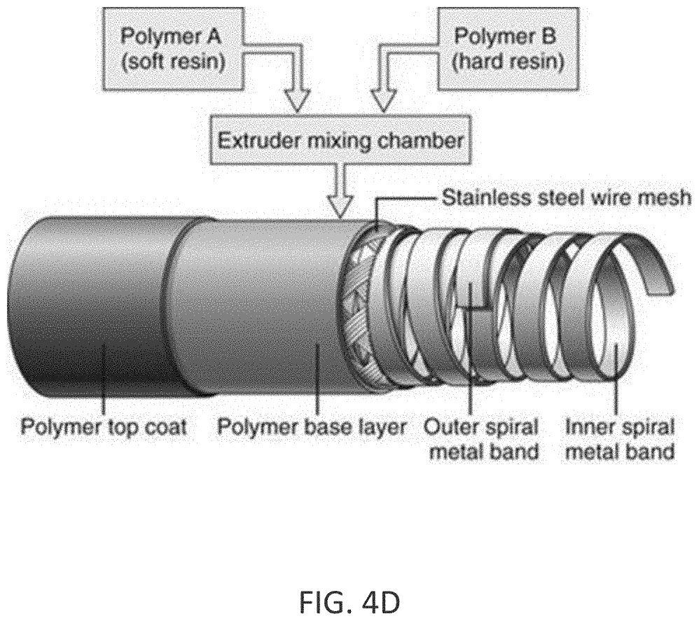

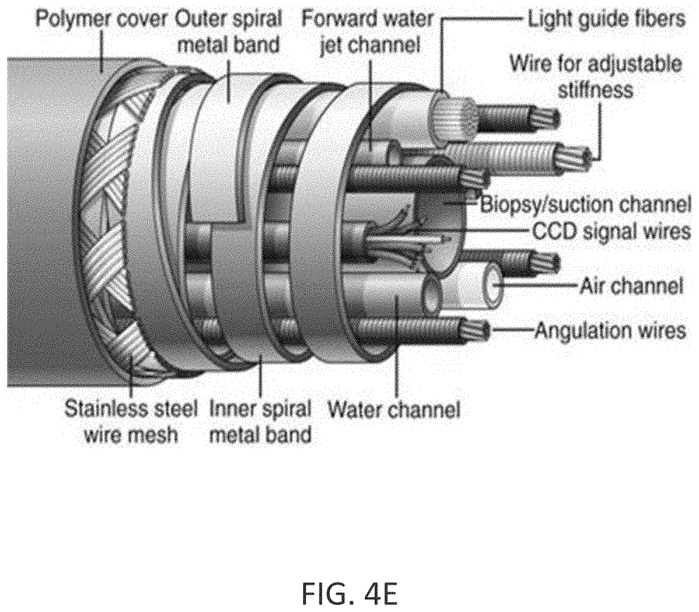

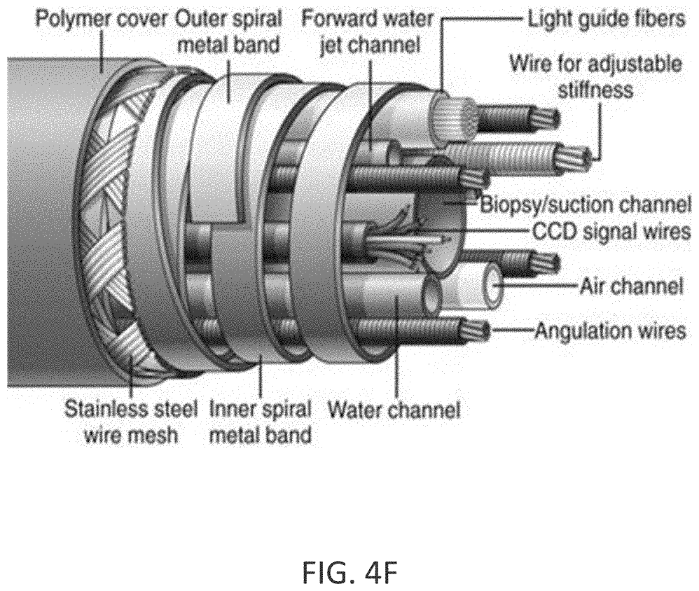

FIG. 4A shows a conventional endoscope inside a colon in a straight position.

FIG. 4B shows a conventional endoscope inside a colon in a looped position.

FIG. 4C shows a conventional endoscope inside a colon in a looped position causing patient discomfort.

FIG. 4D shows a conventional endoscope inside a colon in a looped position causing colon perforation.

The present disclosure relates to the insertion tube sheath of the endoscope. The teachings of the present disclosure are designed to prevent and minimize loop formation by an endoscope during insertion into a flexible hollow organ such as the colon. One or more layers of the insertion tube sheath is replaced with a suitable and comparable mechanical structure made from one more shape memory materials. An exemplary shape memory material used in the present disclosure is Nitinol. In exemplary embodiments of the present disclosure, the Nitinol alloy has an austenite finish temperature of approximately 72° F. (room temperature). In an exemplary embodiment of the present disclosure, the Nitinol alloy has an austenite finish temperature of approximately 98° F. (body temperature).

The Nitinol mechanical structure is designed and programmed (shape setting) such that the mechanical structure assumes a straight position relative to (parallel to) the longitudinal axis of a straight insertion tube sheath (austenite straight position). As such, due to shape memory properties of Nitinol, when the endoscope attempts to form a loop inside a hollow body organ, such as colon, at or above the austenite finish temperature (room temperature/body temperature): Nitinol mechanical structure disposed as one or more layers of the insertion tube sheath counters such looping tendency by attempting to revert to its austenite straight position. This in effect results in stiffening of the insertion tube which prevents/minimizes loop formation. In one exemplary embodiment, a Nitinol alloy with austenite finish temperature of 72° F. is used (as endoscopes are always stored at room temperature outside of the human body). As such, when an endoscope is inserted inside the human body with a body temperature of 98° F. the temperature of the endoscope stays above the exemplary austenite finish temperature of 72° F. at all times. In one exemplary embodiment, a Nitinol alloy has a shape setting temperature of greater than 150° F. that would be well outside of proposed operating range temperature for proposed applications. A great deal of pressure can be produced by preventing the reversion of deformed martensite to austenite. For an exemplary application, a Nitinol alloy with optimal pressure characteristics is used. Optimal pressure characteristics may depend on the target hollow organ to be examined in the proposed application. It is believed that the optimum pressure range for safe examination of the colon is around 10-15 psi.

Exemplary Nitinol Alloy: in one embodiment, a Nitinol alloy has the following properties: a) with austenite finish temperature of about 72° F.; b) a shape setting temperature of >150° F.: and c) optimal pressure characteristics for target hollow organ to be examined.

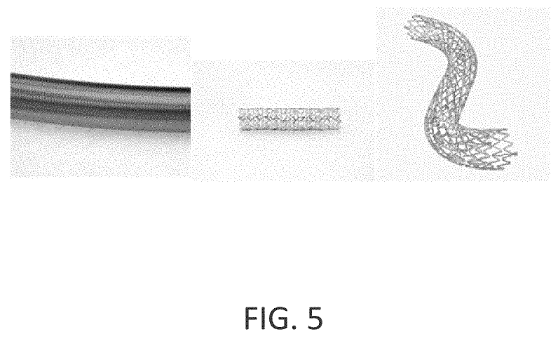

FIG. 4A shows an exemplary embodiment of the present disclosure where stainless steel mesh is replaced with exemplary Nitinol steel mesh. As shown in FIG. 5, the exemplary Nitinol mesh can be designed in one of several ways and as such the mechanical design of the mesh should not be considered limiting the scope of the present disclosure.

FIG. 4B shows an exemplary embodiment of the present disclosure where the inner and/or outer spiral metal band is replaced with exemplary Nitinol inner and/or outer spiral band.

FIG. 4C shows an exemplary embodiment of the present disclosure where the stainless steel mesh and inner spiral band replaced with exemplary Nitinol mesh and exemplary Nitinol inner spiral band, respectively.

FIG. 4D shows an exemplary embodiment of the present disclosure where the stainless steel mesh and outer spiral metal band are replaced with exemplary Nitinol mesh and exemplary Nitinol outer spiral band.

FIG. 4E shows an exemplary embodiment of the present disclosure where the “wire for adjustable stiffness” is replaced with a suitable and comparable wire made with exemplary Nitinol.

FIG. 4F shows an exemplary embodiment of the present disclosure where one or more additional layer of the endoscope sheath is replaced with a suitable and comparable mechanical contraption made with exemplary Nitinol (one or more of wire mesh, stiffening cables, metal bands etc.).

While the present disclosure has been described with reference to one or more exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure.

What is claimed is:

1. An endoscope comprising:
an insertion tube sheath comprising one or more mechanical layers having a mechanical contraption; and
wherein one or more of the mechanical contraptions is made of shape memory material.

2. The endoscope of claim 1 where in the mechanical layers comprises:
a) inner spiral metal band;
b) outer spiral metal band;
c) stainless steel wire mesh; and
d) polymer layer.

3. The endoscope of claim 1, wherein the shape memory material comprises a Nitinol alloy.

4. The endoscope of claim 3, wherein the Nitinol has an austenite finish temperature of around 72° F. (room temperature).

5. The endoscope of claim 3, wherein the Nitinol has an austenite finish temperature of around 98° F. (body temperature).

6. The endoscope of claim 1, wherein the wire mesh is made of Nitinol alloy.
